SEQUENCE LISTING (7 sequences)

SEQ ID NO: 1 - protein, 44 aa, Mus musculus
PLSQPPPVPPSAAAAAGPLAGQPRKTSSSRRNAWGNLSYADLIT

SEQ ID NO: 2 - protein, 652 aa, Mus musculus
MAEAPQVVETDPDFEPLPRQRSCTWPLPRPEFNQSNSTTSSPAPSGGAAANPDAAASLASASAVSTDFMSNLSLLEESEDFARAPGCVAVAAAAAASRGLCGDFQGPEAGCVHPAPPQPPPTGPLSQPPPVPPSAAAAAGPLAGQPRKTSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRGRAAKKKASLQSGQEGPGDSPGSQFSKWPASPGSHSNDDFDNWSTFRPRTSSNASTISGRLSPIMTEQDDLGDGDVHSLVYPPSAAKMASTLPSLSEISNPENMENLLDNLNLLSSPTSLTVSTQSSPGSMMQQTPCYSFAPPNTSLNSPSPNYSKYTYGQSSMSPLPQMPMQTLQDSKSSYGGLNQYNCAPGLLKELLTSDSPPHNDIMSPVDPGVAQPNSRVLGQNVMMGPNSVMPAYGSQASHNKMMNPSSHTHPGHAQQTASVNGRTPPHVVNTMPHTSAMNRLTPVKTPLQVPLSHPMQMSALGRYSSVSSCNGYGRMGVLHQEKLPSDLDGMFIERLDCDMESIIRNDLMDGDTLDFNFDNVLPNQSFPHSVKTTTHSWVSG

SEQ ID NO: 3 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 4 - protein, 722 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPFIDAVWK

SEQ ID NO: 5 - protein, 770 aa, Mus musculus
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMDLTSECATSPM

SEQ ID NO: 6 - protein, 785 aa, Homo sapiens
MSDQDHSMDEMTAVVKIEKGVGGNNGGNGNGGGAFSQARSSSTGSSSSTGGGGQESQPSPLALLAATCSRIESPNENSNNSQGPSQSGGTGELDLTATQLSQGANGWQIISSSSGATPTSKEQSGSSTNGSNGSESSKNRTVSGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQTQYVTNVPVALNGNITLLPVNSVSAATLTPSSQAVTISSSGSQESGSQPVTSGTTISSASLVSSQASSSSFFTNANSYSTTTTTSNMGIMNFTTSGSSGTNSQGQTPQRVSGLQGSDALNIQQNQTSGGSLQAGQQKEGEQNQQTQQQQILIQPQLVQGGQALQALQAAPLSGQTFTTQAISQETLQNLQLQAVPNSGPIIIRTPTVGPNGQVSWQTLQLQNLQVQNPQAQTITLAPMQGVSLGQTSSSNTTLTPIASAASIPAGTVTVNAAQLSSMPGLQTINLSALGTSGIQVHPIQGLPLAIANAPGDHGAQLGLHGAGGDGIHDDTAGGEEGENSPDAQPQAGRRTRREACTCPYCKDSEGRGSGDPGKKKQHICHIQGCGKVYGKTSHLRAHLRWHTGERPFMCTWSYCGKRFTRSDELQRHKRTHTGEKKFACPECPKRFMRSDHLSKHIKTHQNKKGGPGVALSVGTLPLDSGAGSEGSGTATPSALITTNMVAMEAICPEGIARLANSGINVMQVADLQSINISGNGF

SEQ ID NO: 7 - protein, 784 aa, Mus musculus
MSDQDHSMDEVTAVVKIEKDVGGNNGGSGNGGGAAFSQTRSSSTGSSSSSGGGGGQESQPSPLALLAATCSRIESPNENSNNSQGPSQSGGTGELDLTATQLSQGANGWQIISSSSGATPTSKEQSGNSTNGSNGSESSKNRTVSGGQYVVAATPNLQNQQVLTGLPGVMPNIQYQVIPQFQTVDGQQLQFAATGAQVQQDGSGQIQIIPGANQQIIPNEGSGGNIIAAMPNLLQQAVPLQGLANNVLSGQTQYVTNVPVALNGNITLLPVNSVSAATLTPSSQAGTISSSGSQESSSQPVTSGTAISSASLVSSQASSSSFFTNANSYSTTTTTSNMGIMNFTSSGSSGTSSQGQTPQRVGGLQGSDSLNIQQNQTSGGSLQGSQQKEGEQSQQTQQQQILIQPQLVQGGQALQALQAAPLSGQTFTTQAISQETLQNLQLQAVQNSGPIIIRTPTVGPNGQVSWQTLQLQNLQVQNPQAQTITLAPMQGVSLGQTSSSNTTLTPIASAASIPAGTVTVNAAQLSSMPGLQTINLSALGTSGIQVHQLPGLPLAIANTPGDHGTQLGLHGSGGDGIHDETAGGEGENSSDLQPQAGRRTRREACTCPYCKDSEGRASGDPGKKKQHICHIQGCGKVYGKTSHLRAHLRWHTGERPFMCNWSYCGKRFTRSDELQRHKRTHTGEKKFACPECPKRFMRSDHLSKHIKTHQNKKGGPGVALSVGTLPLDSGAGSEGTATPSALITTNMVAMEAICPEGIARLANSGINVMQVTELQSINISGNGF

(19) United States
(12) Patent Application Publication
(10) Pub. No.: US 2011/0098218 A1
(43) Pub. Date: Apr. 28, 2011

(54) MODULATORS OF STAT3 SIGNALLING

(75) Inventors: Weiping Han, Singapore (SG); Guoqing Yang, Singapore (SG)

(73) Assignee: Agency for Science, Technology and Research, Singapore (SG)

(21) Appl. No.: 12/999,595

(22) PCT Filed: Jun. 1, 2009
(86) PCT No.: PCT/SG09/00192

§ 371 Date: Dec. 16, 2010

Related U.S. Application Data
(60) Provisional application No. 61/073,820, filed on Jun. 19, 2008.

(51) Int. Cl. A61K 38/16; G01N 33/53; C07K 5/00; C07K 7/00; C07K 14/47; A61K 38/03; A61P 3/04

(52) U.S. Cl. 514/4.9; 436/501; 435/7.2; 435/7.21; 530/331; 530/330; 530/329; 530/328; 530/327; 530/326; 530/325; 530/324; 530/350

(57) ABSTRACT

The invention relates to methods for identifying compounds which modulate the interaction between STAT3 an SP1. A peptide is provided which is able to bind STAT3 and interfere with the interaction of STAT3 and SP1. The invention provides methods for identifying compounds which are capable of binding to the peptide and thus release interference with the interaction between STAT3 and SP1, as well as methods for identifying inhibitors and enhancers of the STAT3 SP1 interaction. Compounds identified by the methods of the invention are useful in the repression or stimulation of appetite in a patient, useful for the treatment of leptin resistance, obesity and anorexia.

### FIELD OF THE INVENTION

The present invention relates to interactions between STAT3 and SP1 and particularly, although not exclusively, to methods of identifying compounds capable of modulating the interaction between STAT3 and SP1.

### BACKGROUND TO THE INVENTION

Leptin, a hormone secreted from adipose tissue, regulates food intake and energy expenditure (1) by regulating hypothalamic neuron activities. By a saturated transport mechanism, circulating leptin enters brain through the blood-brain barrier to act on at least two classes of neurons: POMC neurons to promote the production of anorexigenic POMC; and NPY/AgRP neurons to down regulate the production and secretion of orexigenic NPY and AgRP (2-4). Leptin exerts its actions through complex signaling pathways upon its binding and activation of the long form leptin receptor (OBRb), but not the other forms of leptin receptors (OBRa, Rc, Rd and Re) (5,6). Activated OBRb turns on Jak2-STAT3 pathway, including STAT3 phosphorylation and translocation into the nucleus, STAT3 binding to target gene promoter/cofactor complexes, and its eventual regulation of target gene promoter activities, e.g. activation of POMC transcription (7).

Plasma and CSF leptin levels are often higher in obese subjects, as expected from their higher fat volume compared with the lean (8). However, leptin fails to effect downstream physiological consequences in these animals due to impairment in the leptin signaling pathways, collectively referred to as leptin resistance (9). The molecular mechanisms underlying leptin resistance are still unclear. One possibility is that increased activity of SOCS3 suppresses STAT3 phosphorylation, and subsequently, prevents STAT3 from translocation into the nucleus and acting on its target genes, based on analysis of DIO mice after 16 weeks of high fat diet feeding (10). Recent studies using DIO mice after 4-5 weeks on a high fat diet showed that the levels of leptin-stimulated STAT3 phosphorylation were comparable to those of lean mice on a chow diet (10, 11). Mice after 4-5 weeks of high fat feeding showed altered metabolism and increased leptin level, indicating that they may be in early stage of leptin resistance (10). The fact that STAT3 phosphorylation was unchanged at this early stage, but was suppressed at late stages of leptin resistance, suggests different molecular mechanisms operating during the early and late stages of leptin resistance. For early stages of leptin resistance, since the level of STAT3 phosphorylation was unaltered, the impairment must lie downstream of STAT3 activation, possibly by a transcription factor.

The transcription factor FoxO1 is a member of forkhead box-containing protein O superfamily, and is a central signaling molecule involved in many aspects of actions, including growth and proliferation as well as metabolic regulation through protein-DNA or protein-protein interactions (14,15). The FoxO1 protein is 655 amino acids in humans, and 652 in mouse (GenBank accession numbers Q12778 (human) and AJ252157 (mouse).

POMC is a key neuropeptide induced by leptin (16). POMC expression is reduced in leptin signaling deficient mouse models, such as ob/ob and db/db mice (17). POMC expression is also reduced in leptin resistant DIO mice (18). Previous studies have shown that leptin-stimulated POMC gene expression is mediated via STAT3 (19).

### SUMMARY OF THE INVENTION

The inventors have discovered that phospho-STAT3 activates POMC promoter activity in response to leptin through a mechanism that requires a SP1 binding site in the promoter of POMC gene. The inventors have also discovered that FoxO1 (SEQ ID NO: 2) binds to STAT3 and prevents STAT3 from interacting with the SP1/POMC promoter complex, and consequently, inhibits STAT3-mediated leptin action. The inventors have determined that this interaction between FoxO1 and STAT3 requires a 44 amino acid region of the FoxO1 protein.

Thus, the inventors have demonstrated for the first time that leptin action can be inhibited at a step downstream of STAT3 activation and translocation into the nucleus, and provides a potential mechanism of leptin resistance in which increased FoxO1 levels antagonize STAT3-mediated leptin signalling.

Also provided is a peptide according to SEQ ID NO: 1 which comprises the FoxO1 binding site for STAT3. Also part of the invention are compounds comprising a peptide having at least 60% sequence identity to SEQ ID NO: 1 and compounds capable of mimicking the interference effect of FoxO1 on the interaction between SP1 and STAT3.

Compounds comprising a peptide having at least 60% sequence identity to SEQ ID NO: 1 can be used to inhibit the interaction between STAT3 and SP1, and thereby inhibit the expression of genes involved in appetite suppression.

Conversely, compounds capable of binding to a peptide having at least 60% sequence identity to SEQ ID NO: 1 can be used to release FoxO1 mediated repression of STAT3/SP1/promoter complex formation by interfering with the interaction between FoxO1 and STAT3. Such compounds can be used to block the repressive effect of FoxO1 on the expression genes which require interaction “between STAT3 and SP1 (”STAT3 SP1 regulated genes“). By maintaining the expression of STAT3 SP1 regulated genes (e.g. the gene encoding POMC), appetite can be suppressed.

Thus, by identifying the amino acid sequence which is essential for the interaction between FoxO1 and STAT3, the inventors have provided methods for identifying compounds capable of stimulating and repressing appetite in a patient in need of treatment. Therapeutic uses of these compounds and pharmaceutical preparations comprising these compounds are part of the present invention.

The invention provides methods, assays and screens for identifying compounds which are capable of modulating the interaction between STAT3 and SP1. In some cases the compounds identified by the methods, assays and screens modulate the interaction by inhibiting the interaction of STAT3 and SP1. In other cases, the test compound may modulate the interaction by enhancing the interaction of STAT3 and SP1.

In a method according to the invention, a STAT3 polypeptide and an SP1 polypeptide are contacted in the presence of a test compound, and the interaction between STAT3 and SP1 is detected. In some cases the test compound is a peptide comprising SEQ ID NO: 1, or comprising a peptide having at least 60% sequence identity to SEQ ID NO: 1. Alternatively, the test compound is a mimetic of the peptide of SEQ ID NO: 1. In other cases the test compound is capable of binding to a peptide which has at least 60% sequence identity to SEQ ID NO: 1.

In cases where the test compound is capable of binding to a peptide comprising SEQ ID NO: 1 or having sequence identity thereto, the interaction between STAT3 and SP1 is assessed in the presence of FoxO1.

In certain methods, compounds capable of modulating the interaction between STAT3 and SP1 are identified by detecting the expression of a STAT3 SP1 regulated gene. Such methods may involve detecting the expression of a reporter gene that is operably linked to the promoter of the STAT3 SP1 regulated gene.

In a first aspect of the invention, a method is provided for identifying modulators of the interaction of STAT3 and SP1, the method comprising:
(a) providing a STAT3 polypeptide;
(b) providing an SP1 polypeptide;
(c) providing a FoxO1 polypeptide;
(d) contacting STAT3 and SP1 polypeptides in the presence of the FoxO1 polypeptide and a test compound; and
(d) detecting binding of STAT3 and SP1;
   wherein the test compound is capable of binding a polypeptide comprising the peptide of SEQ ID NO: 1, or a peptide comprising at least 60% sequence identity to SEQ ID NO: 1.

In a second aspect, a method is provided for identifying modulators of the interaction of STAT3 and SP1, the method comprising:
(a) providing a STAT3 polypeptide;
(b) providing an SP1 polypeptide;
(c) contacting STAT3 and SP1 polypeptides in the presence of a test compound; and
(d) detecting binding of STAT3 and SP1;
   wherein the test compound comprises a peptide comprising at least 60% sequence identity to the peptide of SEQ ID NO: 1, or a mimetic thereof.

In a third aspect, the invention provides a method of identifying compounds capable of suppressing appetite, the method comprising screening a test compound for the ability to bind to a peptide comprising SEQ ID NO: 1, or to a peptide having at least 60% sequence identity to SEQ ID NO: 1.

In some aspects of the invention, binding of STAT3 and SP1 is complex formation.

Certain methods of the invention may involve the step of testing whether the test compound mediates STAT3 SP1 mediated gene expression.

In a fourth aspect, the invention provides an appetite suppressor identified by the methods of the present invention.

In a fifth aspect, the invention provides a medicament comprising an appetite suppressor identified by the methods of the present invention.

In a sixth aspect, the invention provides a method of identifying modulators of the interaction between STAT3 and SP1 comprising the steps of:
(a) providing a cell comprising a STAT3 polypeptide, an SP1 polypeptide, a FoxO1 polypeptide and a STAT3 responsive promoter which is operably linked to a reporter gene;
(b) providing a test compound which is capable of binding to the peptide of SEQ ID NO: 1; and
(c) detecting expression of the reporter gene.

The methods of the invention may comprise the step of:
(d) comparing expression of the reporter gene in step (c) to expression in the absence of the test compound

In a seventh aspect, the methods of the invention include the step of adding leptin.

In an eighth aspect, the invention provides a polypeptide comprising at least 60%, at least 75%, or at least 90% sequence identity to SEQ ID NO: 1. The polypeptide of the invention may comprise between 3 and 100 amino acids or between 3 and 44 amino acids.

In a ninth aspect, the invention provides a mimetic of the polypeptide according to SEQ ID NO: 1 which is capable of disrupting the interaction between STAT3 and SP1.

In a tenth aspect, the invention provides polypeptides or mimetics for use in the manufacture of a medicament for the repression or stimulation of appetite.

Screening Methods

The methods of the present invention may be performed in vitro or in vivo. Where the method is performed in vitro it may comprise a high throughput screening assay.

Test compounds used in the method may be obtained from a synthetic combinatorial peptide library, or may be synthetic peptides or peptide mimetic molecules.

In the methods of the present invention, the STAT3 and SP1 may be obtained from mammalian extracts, produced recombinantly from, bacteria, yeast or higher eukaryotic cells including mammalian cell lines and insect cell lines, or synthesised de novo using commercially available synthesisers. In one arrangement, the STAT3 and SP1 are recombinant. Preferably, the STAT3 and SP1 molecules are human STAT3 and SP1 molecules.

STAT3 (signal transducer and activator of transcription) is a 52 amino acid transcription factor (GenBank ID: AAK17196 (human); AAK17195 (mouse)) that is phosphorylated in response to cytokines and growth factors. Upon phosphorylation, STAT3 dimerises and translocates to the nucleus, where it acts as a transcription factor. STAT3 is responsive to a number of cytokines, hormones and other growth factors, including leptin and IL5. The methods of the present invention utilise a STAT3 polypeptide. STAT3 polypeptides used in the methods of the invention include polypeptides comprising at least 60% sequence identity to SEQ ID NO: 5, or comprise fragments of the polypeptide of SEQ ID NO: 5, or comprising at least 60% sequence identity to fragments of SEQ ID NO: 5.

SP1 (specificity protein) is a transcription factor of approximately 785 amino acids, and comprises a zinc finger DNA binding domain. (GenBank ID: AAC08527 (mouse); AAH43224 (human)). Promoters of certain genes, such as the POMC gene contain SP1 binding sites. The SP1 polypeptides used in the methods of the invention include polypeptides having at least 60% sequence identity to SEQ ID NO: 7, as well as polypeptides comprising fragments of the polypeptide of SEQ ID NO: 7, or which comprise at least 60% sequence identity to fragments of SEQ ID NO: 7. The SP1 polypeptides of the methods of the invention have SP1 DNA binding activity.

Preferably, in the methods of the invention, the STAT3 polypeptides are capable of binding to SP1, and the SP1 polypeptides are capable of binding to STAT3. Preferably, the STAT3 polypeptide is capable of binding to an SP1 polypeptide which is bound to a promoter (an SP1/promoter complex), such as the POMC promoter.

The invention provides methods of identifying compounds which are capable of modulating the interaction of STAT3 and SP1. Modulating the interaction means that the compound is capable of reducing or enhancing the binding of STAT3 and SP1.

In the methods of the invention, STAT3 and SP1 polypeptides are provided and a test compound is added. Binding of the STAT3 and SP1 polypeptides is detected in the presence of the test compound. In some cases, detecting of binding includes detecting the absence of binding. Using the methods of the invention, test compounds which modulate the interaction and binding of the STAT3 and SP1 polypeptides can be identified.

In the methods described, binding may be determined by immunological techniques, including immunoblotting, immunoprecipitation and ELISA.

In certain assays of the invention, a cell (such as a HEK293 cell) is provided which comprises (e.g. expresses) a STAT3 and an SP1 polypeptide. In certain methods, the cell further comprises (e.g. expresses) a FoxO1 polypeptide. A test compound is added to the cell, and the interaction of STAT3 and SP1 is evaluated through detection of a reporter gene which is operably linked to a STAT3 SP1 regulated promoter, such as POMC. In some instances, the reporter gene is luciferase. In some methods, the reporter gene which is operably linked to a STAT3 SP1 regulated promoter is stably or transiently integrated into the genome of a cell. In other methods, the reporter gene which is operably linked to a STAT3 SP1 regulated gene is in a vector.

In certain methods of the invention, vectors comprising the STAT3 and/or SP1 genes are provided. In some methods, a vector comprising a FoxO1 gene is provided. The vector may be an expression vector in which the gene is operably linked. In certain methods, the vectors are provided in a cell. In other methods, the STAT3 and/or SP1 and/or FoxO1 genes are stably integrated into the genome of a cell.

In this specification the term “operably linked” may include the situation where a selected nucleotide sequence and regulatory nucleotide sequence (e.g. a promoter) are covalently linked in such a way as to place the expression of a nucleotide sequence under the influence or control of the regulatory sequence. Thus a regulatory sequence is operably linked to a selected nucleotide sequence if the regulatory sequence is capable of effecting transcription of a nucleotide sequence which forms part or all of the selected nucleotide sequence. Where appropriate, the resulting transcript may then be translated into a desired protein or polypeptide.

Test compounds showing activity in in vitro screens such as high throughput screens can be subsequently tested in screens using cells e.g. in mammalian cells exposed to the candidate modulator, and tested for their ability to modulate the expression of STAT3 SP1 regulated genes.

Test Compounds

A test compound may modulate or interfere with the interaction of STAT3 and SP1 in one of a number of ways. In one arrangement the compound may directly modulate the interaction by binding to one of the molecules, masking the site of interaction. Test compounds preferably comprise a peptide which interacts with the target molecule or an organic compound mimicking the peptide structure (a mimetic).

In some cases, test compounds comprise a peptide having at least 60% sequence identity to SEQ ID NO: 1. In some instances, the peptide comprises more than 65%, more than 70%, more than 75%, more than 80%, more than 85%, more than 90% or more than 95% sequence identity to the SEQ ID NO: 1 peptide. In some cases, the test compound is a fragment of the peptide of SEQ ID NO: 1.

In other cases, the test compound is capable of binding to a polypeptide which comprises a peptide having at least 60% sequence identity to SEQ ID NO: 1. Test compounds which are capable of binding to a polypeptide can be identified through methods known in the art, including co-immunoprecipitation or yeast-2-hybrid screening. Such test compounds will also be capable of binding to FoxO1, or to polypeptides having at least 60% homology to FoxO1.

Optionally, the test compounds of the invention are not STAT3 or SP1 polypeptides, or peptides having high sequence identity to STAT3 or SP1 polypeptides.

The modulating effect of a test compound may be assayed for by measuring an ability to regulate the expression of STAT3 SP1 regulated genes. Such an assay may comprise (a) administering the candidate substance to a test cell, preferably a mammalian cell; and (b) determining the effect of the test compound on the expression of STAT3 SP1 regulated genes.

Binding Affinity

Binding affinity is a measure of the degree to which two components interact. Binding affinity (Kᵢ) can be calculated from the IC₅₀ using the equation of Cheng and Prusoff (Cheng, Y., Prusoff, W. H. (1973) Biochem. Pharmacol. 22, 3099-3108),

Kᵢ=IC₅₀+{1+([Radioligand]/K_{d})}

Where, the IC₅₀ (concentration of the inhibitor that displaces 50% of bound ligand) values are determined by plotting the % specific binding in the Y-axis versus log [molar concentration of protein used] in the X-axis, and K_{d} is the binding affinity of the radioligand to the receptor.

Certain modulators provided by the invention have a high Kᵢ for the peptide of SEQ ID NO: 1. Preferably, such modulators will have a higher Kᵢ for polypeptides comprising SEQ ID NO: 1 than those polypeptides comprising SEQ ID NO: 1 have for STAT3. Such modulators may be useful in the treatment of leptin resistance and obesity.

Interference

Interference of a compound with an interaction relates to the ability of a molecule to interrupt, disrupt or prevent, whether partially or entirely, the normal interaction of STAT3 and SP1 and may be measurable by an altered level of activity of one or more of the normally interacting molecules or by assaying for the presence, absence or partial presence or absence of binding of the normally interacting molecules.

Modulation

Modulation describes the ability of a compound to vary the result of an interaction between interacting substances or molecules. Thus, modulation may be detectable by a change (increase or decrease) in the level of an activity, e.g. in ability to bind to an interacting partner molecule. Modulating compounds may have an enhancing effect or an inhibiting effect on the relevant activity or binding.

Activity

The activity of a given substance or molecule may be measured by assaying for the activity, e.g. luciferase activity can be measured by photon counting. An activity may be a function of the interaction or binding of the given substance, e.g. a modulator peptide comprising SEQ ID NO: 1, with another molecule.

Polypeptides

Polypeptides of the invention include a polypeptide comprising at least 60% identity to SEQ ID NO: 1 and comprising the STAT3 binding site of FoxO1. Polypeptides according to the invention may comprise less than 44 amino acids (e.g. 25, 26, 27, 28, 29, 30, 31, 32, 33, 34, 35, 36, 37, 38, 39, 40, 41, 42 or 43 amino acids), but retain the ability to bind STAT3, and retain at least 60% sequence identity to the polypeptide of SEQ ID NO: 1. Suitable polypeptides may be up to 250 amino acids in length but preferably are 200 amino acids in length or less, or more preferably one of 3-15, 15-30, 30-50, 50-75, 75-100, 100-125, 125-150, 150-175, 175-200, or 200-225 amino acids in length.

In this specification, a modulator polypeptide may be any peptide, polypeptide or protein having an amino acid sequence having a specified degree of sequence identity to SEQ ID NO: 1 or to a fragment of this sequence which is capable of binding to STATS. The specified degree of sequence identity may be from at least 60% to 100% sequence identity. More preferably, the specified degree of sequence identity may be one of at least 65%, 70%, 75%, 80%, 85%, 86%, 87%, 88%, 89%, 90%, 91%, 92%, 93%, 94%, 95%, 96%, 97%, 98% or 99% identity.

Sequence Identity

In certain aspects the invention concerns compounds which are isolated peptides/polypeptides comprising an amino acid sequence having a sequence identity of at least 60% with a given sequence.

Percentage (%) sequence identity is defined as the percentage of amino acid residues in a candidate sequence that are identical with residues in the given listed sequence (referred to by the SEQ ID No.) after aligning the sequences and introducing gaps if necessary, to achieve the maximum sequence identity, and not considering any conservative substitutions as part of the sequence identity. Sequence identity is preferably calculated over the entire length of the respective sequences.

Where the aligned sequences are of different length, sequence identity of the shorter comparison sequence may be determined over the entire length of the longer given sequence or, where the comparison sequence is longer than the given sequence, sequence identity of the comparison sequence may be determined over the entire length of the shorter given sequence.

For example, where a given sequence comprises 100 amino acids and the candidate sequence comprises 10 amino acids, the candidate sequence can only have a maximum identity of 10% to the entire length of the given sequence. This is further illustrated in the following example:

(A)

| | | |
|---|---|---|
| Given seq: | XXXXXXXXXXXXXXX | (15 amino acids) |
| Comparison seq: | XXXXXYYYYYYY | (12 amino acids) |

The given sequence may, for example, be that encoding FoxO1 binding site (e.g. SEQ ID NO: 1).

% sequence identity=the number of identically matching amino acid residues after alignment divided by the total number of amino acid residues in the longer given sequence, i.e. (5 divided by 15)×100=33.3%

Where the comparison sequence is longer than the given sequence, sequence identity may be determined over the entire length of the given sequence. For example:

| (B) | |
|---|---|
| Given seq: | |
| XXXXXXXXXX | (10 amino acids) |
| Comparison seq: | |
| XXXXXYYYYYYZZYZZZZZZ | (20 amino acids) |

Again, the given sequence may, for example, be that encoding FoxO1 binding site (e.g. SEQ ID NO: 1).

% sequence identity=number of identical amino acids after alignment divided by total number of amino acid residues in the given sequence, i.e. (5 divided by 10)×100=50%.

Alignment for purposes of determining percent amino acid sequence identity can be achieved in various ways known to a person of skill in the art, for instance, using publicly available computer software such as ClustalW 1.82. T-coffee or Megalign (DNASTAR) software. When using such software, the default parameters, e.g. for gap penalty and extension penalty, are preferably used. The default parameters of ClustalW 1.82 are: Protein Gap Open Penalty=10.0, Protein Gap Extension Penalty=0.2, Protein matrix=Gonnet, Protein/DNA ENDGAP=−1, Protein/DNA GAPDIST=4.

Identity of nucleic acid sequences may be determined in a similar manner involving aligning the sequences and introducing gaps if necessary, to achieve the maximum sequence identity, and calculating sequence identity over the entire length of the respective sequences. Where the aligned sequences are of different length, sequence identity may be determined as described above and illustrated in examples (A) and (B).

Peptide Derivatives

The peptides of the invention include fragments and derivatives of the FoxO1 binding peptide encoded by SEQ ID NO: 1. Similarly, whilst components used in the methods of the present invention may comprise full-length protein sequences, this is not always necessary. As an alternative, homologues, mutants, derivatives or fragments of the full-length polypeptide may be used.

Derivatives include variants of a given full length protein sequence and include naturally occurring allelic variants and synthetic variants which have substantial amino acid sequence identity to the full length protein.

Protein fragments may be up to 5, 10, 15, 20, 25, 30, 35 or 40 amino acid residues long. Minimum fragment length may be 3, 4, 5, 6, 7, 8, 9, 10, 11, 12, 13, 14, 15, 16, 17, 18, 19, 20 or 30 amino acids or a number of amino acids between 3 and 30.

Mutants may comprise at least one addition, substitution, inversion and/or deletion compared to the corresponding wild-type polypeptide. The mutant may display an altered activity or property, e.g. binding.

Mutations may occur in SEQ ID No: 1 and components containing such fragments may serve the purpose of modulating the activity of the mutant to restore, completely or partially the activity of the wild type polypeptide.

Derivatives may also comprise natural variations or polymorphisms which may exist between individuals or between members of a family. All such derivatives are included within the scope of the invention. Purely as examples, conservative replacements which may be found in such polymorphisms may be between amino acids within the following groups:
(i) alanine, serine, threonine;
(ii) glutamic acid and aspartic acid;
(iii) arginine and leucine;
(iv) asparagine and glutamine;
(v) isoleucine, leucine and valine;
(vi) phenylalanine, tyrosine and tryptophan.

Derivatives may also be in the form of a fusion protein where the protein, fragment, homologue or mutant is fused to another polypeptide, by standard cloning techniques, which may contain a DNA-binding domain, transcriptional activation domain or a ligand suitable for affinity purification (e.g. glutathione-S-transferase or six consecutive histidine residues).

Derivatives of FoxO1 include fragments containing sequence portions having substantial sequence identity to SEQ ID NO: 1 and which are capable of binding STAT3.

Mimetics

The designing of mimetics to a known pharmaceutically active compound is a known approach to the development of pharmaceuticals based on a “lead” compound. This might be desirable where the active compound is difficult or expensive to synthesise or where it is unsuitable for a particular method of administration, e.g. some peptides may be unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled according to its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for in vivo or clinical testing.

With regard to the present invention, having identified a peptide or peptide mimetic in accordance with the method described, the method may further comprise the step of modifying the peptide structure, optionally followed by repeating the contacting and determination steps. This process of modification of the peptide or peptide mimetic may be repeated a number of times, as desired, until a peptide having the desired effect, or level of effect, on binding affinity is identified.

The modification steps employed may comprise truncating the peptide or peptide mimetic length (this may involve synthesising a peptide or peptide mimetic of shorter length), substitution of one or more amino acid residues or chemical groups, and/or chemically modifying the peptide or peptide mimetic to increase stability, resistance to degradation, transport across cell membranes and/or resistance to clearance from the body.

Therapeutic Applications

Compounds of the present invention or identified by methods of the present invention may be used stimulate or repress appetite in animals in need of treatment. Preferably, the animal undergoing treatment is a human patient in need of such treatment. More particularly, the compounds may be used in either stimulating or repressing appetite.

Enhancers of the interaction between STAT3 and SP1 may be useful in the treatment of obesity and leptin resistance and in the suppression of appetite by enhancing the interaction of STAT3 with the SP1/promoter complex, and thereby enhancing expression of leptin regulated genes such as POMC.

Inhibitors of the STAT3 SP1 interaction may be useful for stimulating appetite. Inhibitors may be useful in the treatment of anorexia and other eating disorders. Inhibitors of the STAT3 SP1 interaction impair the ability of STAT3 to bind the SP1/promoter complex, and thereby prevent STAT3 from promoting the expression of genes e.g. POMC in response to leptin.

Compounds of the invention may be formulated as pharmaceutical compositions for clinical use and may comprise a pharmaceutically acceptable carrier, diluent or adjuvant. The composition may be formulated for topical, parenteral, intravenous, intramuscular, intrathecal, intraocular, subcutaneous, oral, inhalational or transdermal routes of administration which may include injection. Injectable formulations may comprise the selected compound in a sterile or isotonic medium.

Formulating Pharmaceutically Useful Compositions and Medicaments

In accordance with the present invention methods are also provided for the production of pharmaceutically useful compositions, which may be based on a substance or test compound so identified. In addition to the steps of the methods described herein, such methods of production may further comprise one or more steps selected from:
(a) identifying and/or characterising the structure of a selected substance or test compound;
(b) obtaining the substance or compound;
(c) mixing the selected substance or compound with a pharmaceutically acceptable carrier, adjuvant or diluent.

For example, a further aspect of the present invention relates to a method of formulating or producing a pharmaceutical composition for use in the treatment of leptin resistance and obesity, the method comprising identifying a compound or substance that promotes or inhibits interaction of STATS and SP1, in accordance with one or more of the methods described herein, and further comprising one or more of the steps of:
(i) identifying the compound or substance; and/or
(ii) formulating a pharmaceutical composition by mixing the selected substance, or a prodrug thereof, with a pharmaceutically acceptable carrier, adjuvant or diluent.

Certain pharmaceutical compositions formulated by such methods may comprise a prodrug of the selected substance wherein the prodrug is convertible in the human or animal body to the desired active agent. In other cases the active agent may be present in the pharmaceutical composition so produced and may be present in the form of a physiologically acceptable salt.

### BRIEF DESCRIPTION OF THE FIGURES

Embodiments and experiments illustrating the principles of the invention will now be discussed with reference to the accompanying figures in which:

FIG. 1: STAT3-mediated leptin regulation of POMC promoter activity in a cell-based system.

(A) Diagram (upper panel) depicts leptin receptor constructs stably expressed in the recombinant HEK 293 cells. The solenoid represents plasma membrane (PM). OBRa and OBRb share identical extracellular sequences, including leptin binding sites (shaded area near PM). “Y” denotes the tyrosine residues implicated in leptin signaling, and is present only in OBRb. Both constructs are Myc-tagged at their C termini (black area). Lower panel shows expression of leptin receptors in the 293 cells lines. Leptin receptors from lysates of 293-OBRa, 293-OBRb and control were concentrated by using leptin-coupled CNBR-activated Sepharose beads, and their expression examined by using Myc antibody. (B) 125I-leptin was mixed with control, 293-OBRa or 293-OBRb cells with (white column) or without (gray column) the addition of excessive amount of unlabeled leptin. The cells were washed and radioactivity counted. Results are mean±SEM, and represent 3 independent experiments: *p<0.01. (C) 293-OBRa and 293-OBRb were transfected with pXJ40-Flag-mSTAT3. The cells were lysed and subjected to 8% SDSPAGE after 30 min of leptin or mock treatment. Both phospho-STAT3 and pan-STAT3 antibodies were used for protein detection. Note that phospho-STAT3 signal was detected only in leptin-treated 293-OBRb cells, whereas pan-STAT3 signal was evident in all samples. (D) 293-OBRa or 293-OBRb was transfected with pXJ40-Flag-mSTAT3, pGL3-POMC and pCMV-Renilla. 20 hr after leptin treatment, the cells were harvested and lysed, and Firefly luciferase activity of the lysate was measured and normalized to Renilla luciferase activity. Results are mean±SEM, and represent 3 independent experiments. *p<0.01.

FIG. 2. FoxO1 inhibits leptin-induced POMC promoter activity.

(A) 293-OBRb cells were transfected with the same amount of pXJ40-Flag-mSTAT3 and pGL3-POMC, plus increasing amount of pcDNA3- Flag-mFoxO1, as indicated by solid bars for POMC and STAT3, and solid staircase for FoxO1. A promoter-less pGL3-basic was transfected in place of pGL3-POMC as negative control (lane 1 and 2). 20 hr after leptin treatment, the cells were harvested for immunoblotting using antibodies against phospho-STAT3, pan-STAT3, or FoxO1. Tubulin was included to indicate equal loading among all the samples. Note that FoxO1 expression increased proportionately with increasing amount of transfected pcDNA3-Flag-mFoxO1. (B) Similarly-treated cells as in (A) were lysed in passive lysis buffer, and their Firefly luciferase activity was measured and normalized to Renilla luciferase activity. The assay was repeated 3 times in triplicate. Results represent mean±SEM of one such assay.

FIG. 3. High level of FoxO1 does not interfere with STAT3 phosphorylation or STAT3 translocation into nucleus.

(A) 293-OBRb cells were transfected with the same amount of pXJ40-Flag-mSTAT3 and pGL3-POMC, plus increasing amount of pcDNA3-Flag-mFoxO1, as indicated by solid bars for POMC and STAT3, and solid staircase for FoxO1. 30 min after leptin treatment, the cells were lysed in hypotonic buffer followed by centrifugation and high salt treatment to separate nuclear and cytoplasmic fractions, as described in Experimental Procedures. Equal amount of nuclear proteins was loaded based on Bradford measurement, and evidenced by tubulin signals (lower panel). Immunoblots show nuclear proteins probed with antibodies against phospho-STAT3 (top panel), or FoxO1 (middle panel). (B) 293-OBRb cells were transfected with pXJ40-Flag-mSTAT3 alone (a, c) or together with pcDNA-Myc-mFoxO1 (b, d). After leptin (c, d) or mock (a, b) treatment, cells were fixed, permeablized, and probed with antibodies against STAT3 (green) and FoxO1 (red). STAT3 signals were mostly cytoplasmic without leptin treatment, but concentrated in the nucleus in leptin-treated samples.

FIG. 4. Essential DNA element in the POMC promoter (−646 to +65) mediating leptin regulation of POMC transcriptional activity.

(A) Diagram of wildtype (WT) POMC promoter and deletion mutants. Details of all the mutants were described in FIG. 8. (B) 293-OBRb cells were transfected with pXJ40-Flag-mSTAT3, pGL3-POMC and pCMV-Renilla. 20 hr after leptin treatment, the cells were lysed in passive lysis buffer. Firefly luciferase activity was measured and normalized to Renilla luciferase activity. Results are presented as mean±SEM, and are a representative of at least 3 independent experiments of triplicate.

FIG. 5. Mutation of SP1 binding site abolishes leptin regulation of POMC promoter activity.

(A) Diagram of pGL3-POMC construct showing sequence of the essential DNA element (−138 to −88) mediating leptin regulation of POMC promoter activity. EMSA probes containing putative SP1 binding site (Probe 1) or point mutations (Probe 2) were synthesized as described in Experimental Procedures. Base mutations were highlighted in red. (B) EMSA with probe 1 or 2 was carried out using nuclear extracts of 293-OBRb cells expressing Flag-mSTAT3. A nuclear protein bound to Probe 1 (arrow, lane 1 and 2), but not Probe 2 (lane 3 and 4). The protein binding was specifically inhibited by an SP1 antibody (lane 5 and 6). Samples from two independent experiments were loaded to illustrate reproducibility. (C) Diagram of WT POMC promoter and SP1 binding site mutants. Details of the mutants were described in FIG. 8. Base mutations were highlighted in red. (D) 293-OBRb cells were transfected with pXJ40-Flag-mSTAT3 and pCMV-Renilla, plus pGL3-POMC, mutant 12 or 13. 20 hr after leptin treatment, the cells were lysed in passive lysis buffer. Firefly luciferase activity was measured and normalized to Renilla luciferase activity. Results are presented as mean±SEM, and are a representative of at least 3 independent experiments of triplicate.

FIG. 6. FoxO1 inhibits STAT3-SP1 complex formation by binding to STAT3.

(A) 293-OBRb cells were transfected with pXJ40-Flag-mSTAT3. After treatment with leptin or vehicle, the cells were lysed in lysis buffer. Cell lysate was incubated with SP1 antibody or control IgG. 5% of cell lysate used in co-IP samples were loaded as input. (B, C) 293-OBRb cells were transfected with pXJ40-Flag-mSTAT3 and pcDNA3-Myc-mFoxO1. After leptin treatment, the cells were lysed in lysis buffer. Cell lysate was incubated with 1 pg of anti-Flag (B), anti-Myc (C), or control IgG. Immunoblot (IB) using antibodies against either Myc (B) or Flag (C) revealed STAT3-FoxO1 interaction. (D) 293-OBRb cells were transfected with the same amount of pXJ40-Flag-mSTAT3 and increasing amount of pcDNA3-MycmFoxO1 as indicated by solid bar (STAT3) and staircase (FoxO1). 30 min after leptin treatment, nuclear proteins were isolated from these cells and subjected to IP using Flag antibody. IB with either anti-Myc or anti-SP1 revealed that the amount of SP1 decreased with increasing amount of FoxO1.

FIG. 7. Potential mechanism of leptin regulation of POMC promoter activity and its inhibition by FoxO1.

(A) Upon leptin binding to OBRb, STAT3 is phosphorylated. Activated STAT3 translocates into the nucleus and activates POMC promoter activity through its interaction with SP1-POMC promoter complex. (B) With increasing amount of FoxO1 expression, FoxO1 binds to phosphorylated STAT3 in the nucleus, and prevents STAT3 from interacting with the SP1-POMC promoter complex, and consequently, inhibits STAT3-mediated leptin activation of POMC promoter.

FIG. 8: DNA constructs

DNA constructs used in this study, including truncation and mutation constructs based on pGL3-POMC.

FIG. 9: Primers.

Primers used in the generation of the DNA constructs described in FIG. 8.

FIG. 10. FoxO1 constructs generated to identify the STAT3 binding site on FoxO1.

FoxO1 is a 652 aa protein. A series of C-terminal deletion constructs were made and tested their interaction with STAT3 by coimmunoprecipitation (+ or − indicates whether construct bound STAT3 in coimmunoprecipitation). FoxO1⁽¹⁻¹⁶⁷⁾ and other longer FoxO1 mutants were able to bind to STAT3, while FoxO1⁽¹⁻¹²³⁾ failed to bind to STAT3. This suggests that the region between 123-167 is important for STAT3 interaction. FoxO1⁽¹⁻¹²³⁾⁻⁽¹⁶⁸⁻⁶⁵²⁾ is a deletion construct which does not contain the region identified in the previous C-terminal deletion constructs. As a control, we also generated a FoxO1 mutant that does not contain the region between 168-241, FoxO1⁽¹⁻¹⁶⁷⁾⁻⁽²⁴²⁻⁶⁵²⁾. Co-IP experiments using the above two deletion constructs confirmed C-terminal deletion results that the region corresponding to amino acids 124-167 was necessary for STAT3 interaction.

FIG. 11: Sequences

Sequences of polypeptides described in the application.

### DETAILED DESCRIPTION OF THE INVENTION

The details of one or more embodiments of the invention are set forth in the accompanying description below including specific details of the best mode contemplated by the inventors for carrying out the invention, by way of example. It will be apparent to one skilled in the art that the present invention may be practiced without limitation to these specific details.

### EXAMPLES

Experimental Procedures

DNA Constructs: The POMC promoter-luciferase construct (pGL3-POMC) was a generous gift from Dr. Domenico Accili (Columbia University, USA), pcDNA3-Flag-mFoxO1 from Dr. Fukamizu (Japan), pN3-SP1 FL-complete from Dr. Suske (Germany). pXJ40-flag-STATS was described previously (20). All the other DNA constructs and primers used in this study, including truncation and mutation constructs based on pGL3-POMC, are described in tables 1 and 2.

Cell Culture and Luciferase Assay: Flp-InHEK293 stable cell lines over-expressing OBRa(293-OBRa) or OBRb (293-OBRb) were described previously (21). Cells were cultured in Dulbecco's minimal essential medium (DMEM, Invitrogen) containing 10% fetal bovine serum (FBS) in a 37° C. incubator with 5% CO2. One day after plating, cells were transfected with relevant DNA constructs using Fugene 6 (Roche). 16 hr later, transfected cells were serum-starved for 5 hr before they were treated with recombinant leptin (Invitrogen) or vehicle for 20 hr. Cells were then washed with PBS and lysed in 200 μl of 1× passive lysis buffer included in Dual-Luciferase Reporter Assay System (Promega). Luciferase activity was measured from cell extracts on aluminometer (Molecular Devices). The firefly luciferase activity was normalized against Renilla luciferase activity.

Detection of OBRa and OBRb in 293 stable cell lines: 293-OBRa and 293-OBRb cells were harvested and lysed with lysis buffer, and incubated with leptin-coupled CNBR-activated Sepharose beads (Sigma) overnight. After repeated washing with lysis buffer, the beads with pulled down proteins were subjected to SDS-PAGE. Leptin receptor expression was examined by using Myc antibodies.

Leptin Binding to Stable HEK293 Cells: This was performed in six-well plates as previously described (21). Briefly, 293-OBRa or 293-OBRb cells were grown to ˜90% confluence and washed with PBS. Cells were incubated with approximately 60,000 cpm of murine recombinant 125I-leptin (Perkin-Elmer) alone, or 125I-leptin with excessive amount of unlabeled leptin (2 μg/well) for 6 hr at 4° C. in a final volume of 1 ml PBS supplemented with 1% (w/v) BSA (fraction V, Sigma). At the end of incubation, unbound 125I leptin was removed by two PBS washes. 1 ml of 1N NaOH was then added, and radioactivity in the lysate was measured using a Wizard 1470 Automatic Gamma Counter (Perkin-Elmer).

Nuclear extract preparation from 293 cells: Cells after treatment with leptin or vehicle were washed twice and collected in cold PBS. The cell suspension was centrifuged at 1,300 rpm for 5 min. The resulting pellet was resuspended with hypotonic buffer (20 mM HEPES pH 7.9, 10 mM KCl, 1 mM EDTA, 1 mM Na3VO4, 10% glycerol, 0.2% NP-40, 20 mM NaF, 1 mM DTT and 1× complete protease inhibitor (Roche)), and rocked at 4° C. for 10 min. The mixture was then centrifuged at 13,000 rpm for 30 sec, and high salt buffer (20% glycerol, 420 mM NaCl, 1 mM Na3VO4, 1 mM DTT and 1× complete protease inhibitor in hypotonic buffer without NP-40) was added to resuspend the pellet. After 40 min rocking, the mixture was centrifuged at 13,000 rpm for 10 min at 4° C. The supernatant was collected as the nuclear extract. Co-IP: 1) For STATS-SP1 interaction, 293-OBRb cells were transfected with pXJ40-Flag-mSTAT3, and followed by leptin treatment. Nuclear extracts were prepared from the cells and incubated with SP1 antibody for immunoprecipitation (IP). Immunoblotting of the immunoprecipitation was performed using phospho-STAT3 antibody (Cell Signaling). 5% of cell lysate used in each colP sample was loaded as input. 2) For STAT3-FoxO1 interaction, 293-OBRb cells transfected with expression vectors of pXJ40-Flag-mSTAT3 and pcDNA3-Myc-mFoxO1 were serum-starved and treated with leptin (50 nM) for 30 min, and then lysed in lysis buffer (20 mM Tris-Cl, pH 7.5, 150 mM NaCl, 1% Triton-X-100, 10 mM NaF, 1 mM EDTA, 1 mM Na3VO4, 1 mM PMSF, supplemented with protease inhibitors). ˜500 μg cell lysate was incubated for 2 hr with 1 μg Flag (Sigma), Myc (Santa Cruz Biotechnology) antibodies, or control IgG, respectively, followed by IP with protein A+G Sepharose beads (Sigma) for 1 hr.

The immunoprecipitates were washed 4times in lysis buffer and subjected to SDS-PAGE and immunoblotting with antibodies against Flagor Myc. 5% of cell lysate used in each colP sample was loaded as input. 3) For FoxO1 effects on STAT3-SP1 interaction, pXJ40-Flag-mSTAT3 and increasing amount of pcDNA3-Myc-mFoxO1 were transfected into 293-OBRb cells. Cells were harvested for nuclear fractionation after leptin treatment. Binding of STAT3 to SP1 in nuclear extracts was examined by IP with Flag antibody and IB with Myc (for STAT3) and SP1 antibodies.

Immunoblotting: Cells were lysed in 1× cell lysis buffer (Cell Signaling) containing 1 mM PMSF. Lysate was incubated on ice for 20 min with gentle rocking and centrifuged at 20,000×g for 10 min at 4° C. Equivalent amount of samples were analyzed by SDS-PAGE and immunoblotting using antibodies against phospho-STAT3 (Cell Signaling Technology); pan-STAT3, FoxO1, SP1 and Myc (Santa Cruz Biotechnology); Flag (Sigma); and Myc (polyclonal, Upstate). EMSA: Two pairs of oligonucleotides: wild type (GAG GCC CGC CGC CCC CCT and GAA GGGGGG CGG CGG GC) and SP1 binding site mutant sequence (GAG GCT TGT TGC CCC CCT and GAA GGG GAA CAA CGG GC) were annealed, and about 100 ng of the probes were labelled with 50 μCi of 32P dCTP by klenowexo-(NEB). After labelling, the probes were purified by using G-50column, and radioactivity was measured with LS6500 Multi-Purpose Scintillation Counter (Beckmam Coulter). 5 μg of nuclear protein was incubated with the probe with 20,000 cpm in DNA-protein loading buffers (50 mM NaCl, 10 mM TrisCl pH 7.5, 0.5 mM EDTA, 1 mM MgCl2, 4% Ficoll, 0.5 mM DTT and 1× complete protease inhibitor) in a total volume of 12 μl at room temperature for 15 min. The mixture was resolved by 4% PAGE gel in 0.5× TBE, and the gel dried at 80° C. by using a gel dryer (Bio-rad) for 2 hr. Super sensitive X-ray film (Kodak) was exposed for 48 hr at −80° C. and then developed.

Immunocytochemistry: 293-OBRb cells were transfected with relevant plasmids one day after they were plated on poly-lysine coated coverslips. After leptin or mock treatment, the cells were washed with PBS, fixed in PBS containing 4%paraformaldehyde for 10 min, permeabilized in PBS containing 0.5% triton X-100 for 10 min, and blocked in ICC buffer (3% BSA, 3% goat serum, and 0.15% triton X-100 in PBS) for 1 hr at room temperature. The cells were then probed by using STAT3 and FoxO1 antibodies, and fluorescence conjugated secondary antibodies (Invitrogen). Coverslips were mounted on slides and sealed for observation by confocal microscopy.

Statistical Analysis: The data were presented as means±S.E.M. Comparisons of data were made using two-tailed Student's t-test for independent data. The significance limit was set at p<0.05.

### Example 1

### Leptin Regulation of POMC Promoter Activity Via STAT3 Activation

To understand how STAT3 signaling may be inhibited downstream of its activation, a cell-based system was established to investigate how STAT3 mediates leptin regulation of gene expression. The cell-based system includes stable expression of OBRb, and transient expression of Firefly luciferase under the POMC promoter.

POMC promoter was chosen to study STAT3-mediated leptin regulation because: 1. POMC is a key anorexigenic neuropeptide that is regulated by leptin and STAT3 (19), 2. POMC expression is reduced in leptin-resistant DIO mice (18).

Establishment of Cell Based System

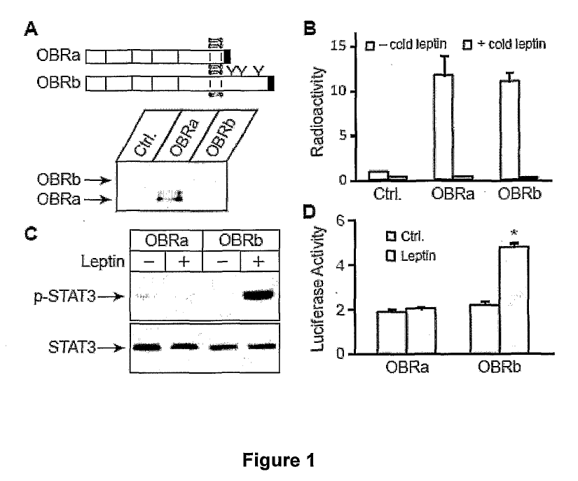

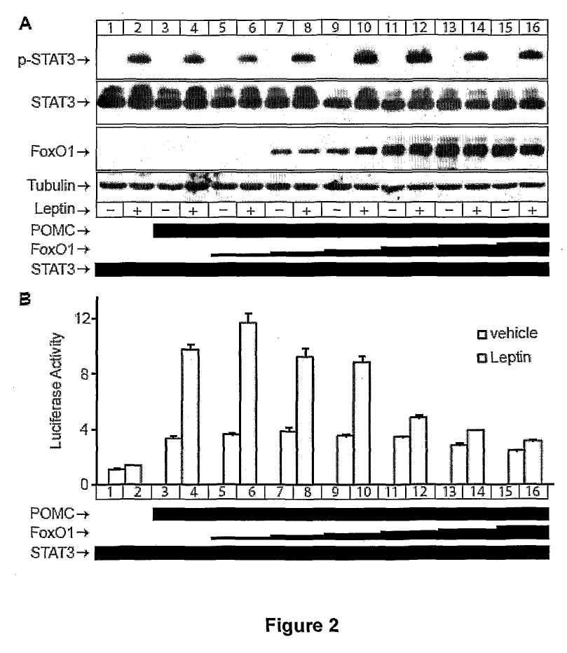

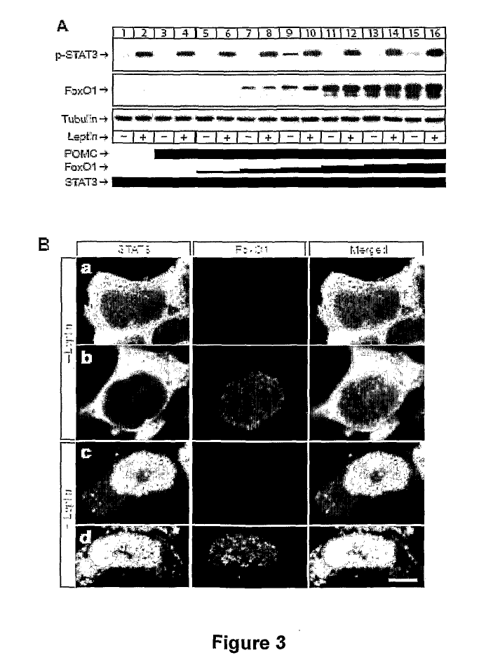

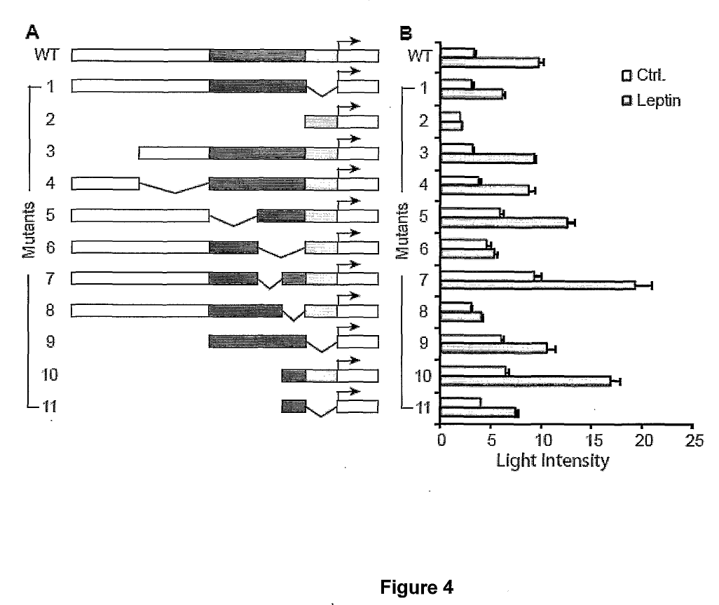

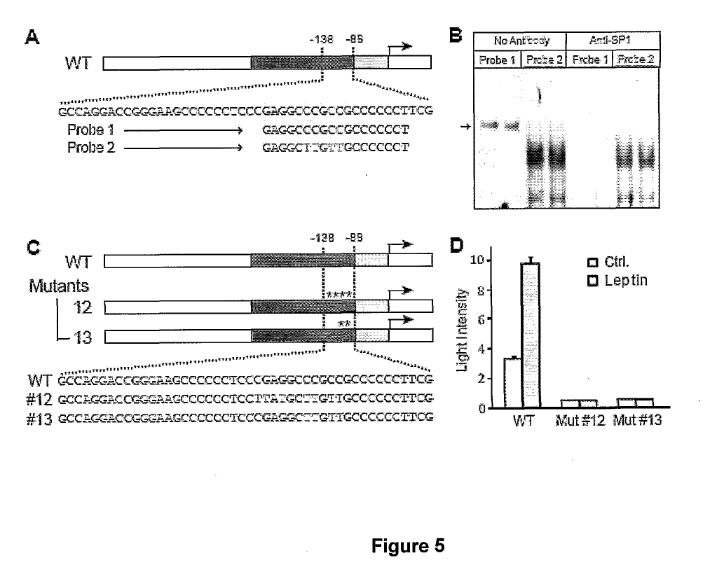

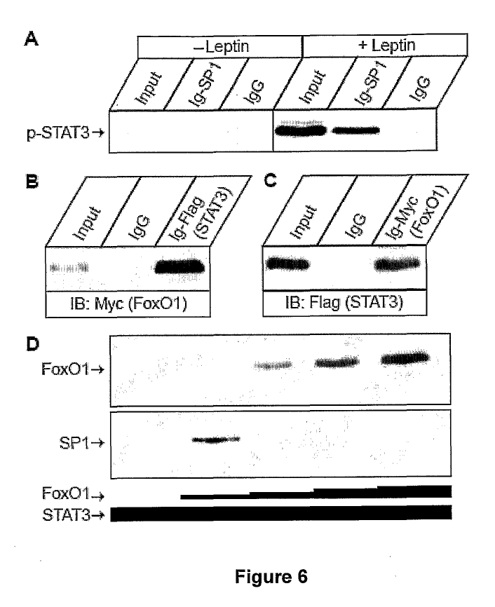

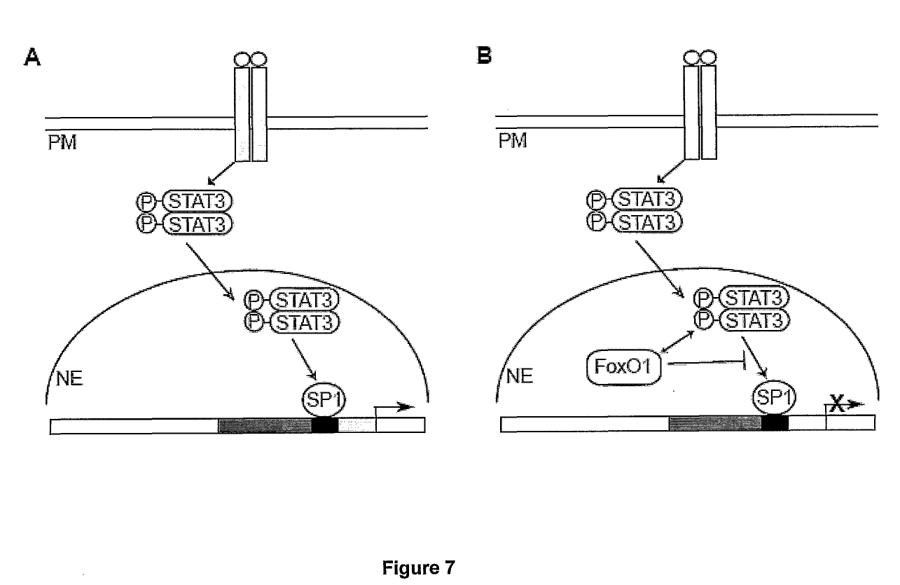

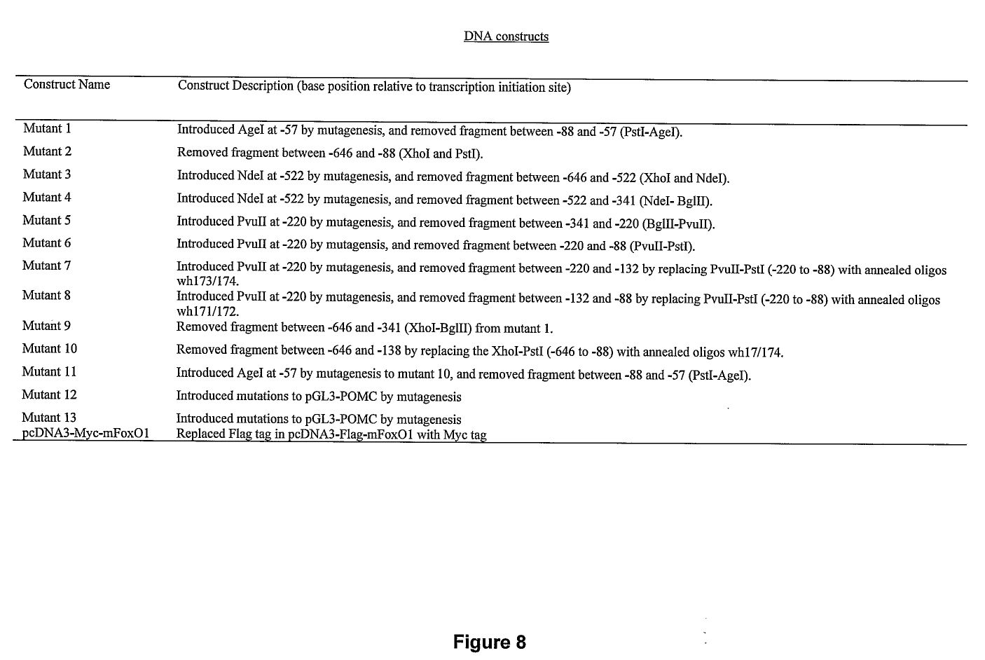

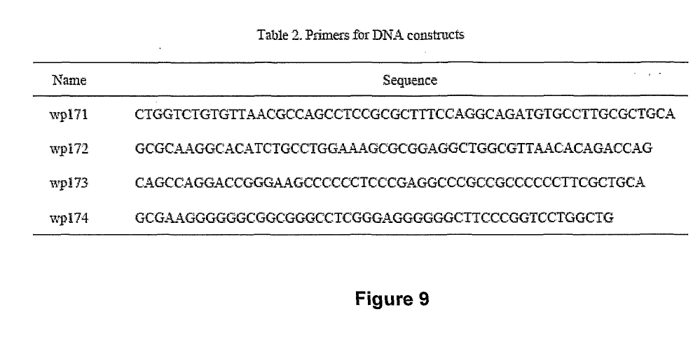

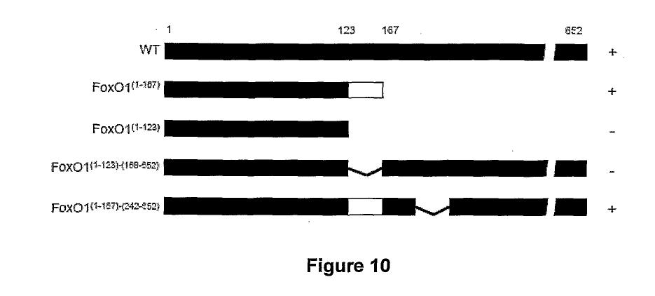

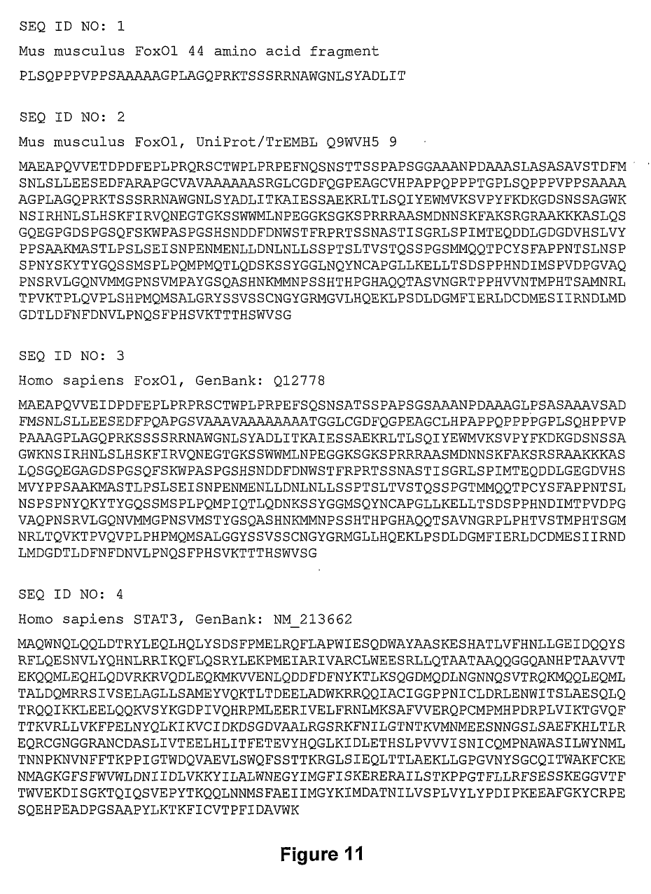

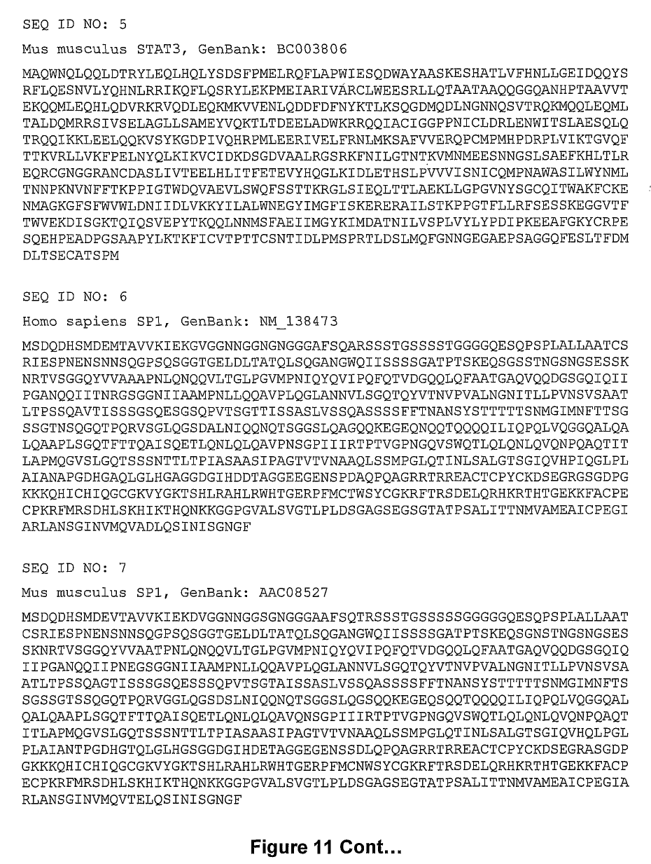

Leptin regulates energy homeostasis mainly through its central action by binding and activating the long form leptin receptor OBRb, but not the other forms (5,6). HEK 293 cell lines with stable expression of OBRb (293-OBRb) were established as an in vitro system to study leptin regulation of POMC promoter activity. HEK 293 cells over-expressing OBRa (293-OBRa) was used as a negative control. In these cell lines, only a single copy of the gene construct with C-terminal Myc tagging (FIG. 1A, upper panel) was integrated into the genome to ensure consistent expression level of respective receptors.

Since expression level of the receptors in the stable cell lines was not abundant enough for direct detection from cell lysate by Western blotting, the proteins were concentrated by using leptin-coupled beads. OBRa or OBRb could be detected only in respective stable cell lines, but not the control (FIG. 1A, lower panel). To further confirm the expression of OBRa or OBRb, and to validate their proper localization and orientation on the cell surface in these cell lines, cells were incubated with 125I-labeled leptin in the presence or absence of excessive unlabeled leptin. 125I-labeled leptin could bind both 293-OBRa and 293-OBRb to a similar extent (FIG. 1B). Radioactivity of 125I-labeled leptin, indicative of leptin binding, was not detectable in control cells or in the presence of excessive unlabeled leptin (FIG. 1B).

A plasmid containing the luciferase gene driven by POMC promoter was introduced into 293-OBRb and the control 293-OBRa by transient transfection to test whether 293-OBRb cells could be used as an in vitro system to study leptin regulation of promoter activity. We used the POMC promoter containing −646 to +65 of the POMC gene, as full promoter activity requires no more than 480 by DNA fragment upstream of transcription initiation site (13,22).

Leptin treatment induced STAT3 phosphorylation only in 293-OBRb cells (FIG. 1C). Similarly, leptin stimulated luciferase activity was only observed in 293-OBRb, but not in 293-OBRa cells (FIG. 1D), consistent with previous findings that only OBRb is capable of leptin signal transduction (6). Taken together, 293-OBRb was a suitable system in studying POMC promoter activity regulation by STAT3-mediated leptin signaling.

### Example 2

### FoxO1 Inhibits STAT3-Mediated POMC Activity

In early stages of leptin resistance, levels of phospho-STAT3 are comparable in mice on high fat diet with those on normal chow diet, indicating that impairment of leptin signalling lies downstream of STAT3 activation (10). To mimic the early stages of leptin resistance, in which STAT3 phosphorylation was not reduced, 293-OBRb cells were transfected with the amount of STAT3 that resulted in maximal level of leptin induced POMC promoter activation (data not shown).

An increasing amount of FoxO1 cDNA was introduced on the background of constant STAT3 level (FIG. 2A) to test whether FoxO1 could interfere with leptin-induced POMC promoter activity. FoxO1 expression levels increased proportionately with increasing amounts of cDNA used for transfection (FIG. 2A). Although leptin-induced STAT3 phosphorylation was not affected by increasing FoxO1 expression, leptin-regulation of POMC promoter activity, as indicated by luciferase activity, was abolished at high expression levels of FoxO1 (FIG. 2B). Leptin-regulation of POMC promoter activity was not affected when increasing amount of a similar-sized control protein was introduced (data not shown). These data demonstrate that high levels of FoxO1 could interfere with leptin signalling, and suggest FoxO1 acted at a step downstream of STAT3 activation.

### Example 3

### FoxO1 Inhibits STAT3 Action in the Nucleus

To further delineate at which step increasing FoxO1 affected leptin signalling, we tested whether FoxO1 suppressed STAT3 translocation into nucleus after leptin activation. 293-OBRb cells were transfected with increasing amount of FoxO1 cDNA on the background of constant STAT3 level, and nuclear and cytoplasmic components were separated by fractionation. As expected, FoxO1 protein levels increased in the nuclear fraction (FIG. 3A, second panel) with increasing amount of FoxO1 cDNA; while phosphorylated STAT3 in the nucleus remained at the same level regardless of FoxO1 expression levels (FIG. 3A, first panel). To directly visualize the effects of FoxO1 on leptin-induced STAT3 activation and translocation into the nucleus, we performed immunocytochemistry and confocal microscopy were performed on 293-OBRb cells expressing STAT3 alone or STAT3 plus FoxO1. STAT3 signals were mostly cytoplasmic without leptin stimulation (FIG. 3B, panel a & b), but concentrated in the nucleus in leptin-treated samples (FIG. 3B, panel c & d). The extent of STAT3 translocation into the nucleus as indicated by the STAT3 signal in the nucleus, was indistinguishable between cells with and those without FoxO1 (FIG. 3B, panel c & d). These data showed that FoxO1 affected neither leptin-induced STAT3 phosphorylation nor the subsequent STAT3 translocation into the nucleus, and indicated that FoxO1-mediated inhibition of leptin-regulation of POMC promoter activity happened downstream of STAT3 translocation into the nucleus, i.e., high level of FoxO1 prevents STAT3 from activating the POMC promoter in the nucleus.

### Example 4

### Essential DNA Fragment for Leptin Induced POMC Promoter Activity

To understand how FoxO1 inhibits STAT3-mediated POMC promoter activation, the mode of interaction between STAT3 and POMC promoter was investigated. A series of mutants with deletion in the promoter region of POMC (mutants #1-11, FIG. 4A) on the background of pGL3-POMC (WT, FIG. 4A) were made to determine the essential sequence for STAT3-mediated leptin activation of POMC promoter activity. Mutant constructs, along with pGL3-POMC, were separately introduced into 293-OBRb cells, and luciferase activity of various POMC promoter constructs with or without leptin treatment was determined. Deletion mutants without DNA fragment between −138 and −88 (#2, 6 and 8) resulted in the loss of leptin regulation of POMC promoter activity, whereas all the mutants containing this fragment retained leptin regulation, including mutant #11 containing only this DNA fragment (−138 to −88) fused directly upstream of POMC promoter TATA box (FIG. 4B), indicating that a DNA binding element critical to leptin-enhanced POMC promoter activity lies between −138 and −88 by upstream from the transcription initiation site.

### Example 5

### SP1 Binding Element is Necessary for POMC Promoter Activity

To identify the DNA fragment of POMC promoter responsible for normal leptin response the structure of POMC promoter was investigated.

Sequence analysis revealed that the DNA element between −138 and −88 contained a consensus binding sequence to SP1 (FIG. 5A), a constitutive transcription factor present in most cell types (23). To verify whether the putative SP1 binding site interacts with SP1, probe 1, corresponding to the original sequence, and probe 2, containing mutations in the putative SP1 binding site (FIG. 5A) were synthesised, and EMSA was performed with nuclear extracts from 293-OBRb cells. A nuclear protein bound specifically to probe 1, but not to probe 2, and the binding to probe 1 was specifically inhibited by a SP1 antibody (FIG. 5B), but not by STAT3 or FoxO1 antibodies (data not shown). These data indicated that the bound nuclear protein was SP1, and SP1 and probe 1 formed a specific complex. To examine the potential function of SP1 binding site in POMC promoter activity, mutant #12 and 13, were generated which contained point mutations within SP1 binding site and adjacent sequence (mutant #12) or within SP1 binding site only (mutant #13) (FIG. 5C). Functional analysis of these mutants in 293-OBRb cells revealed the promoter activity of both mutants as well as their regulation by leptin were abolished (FIG. 5D), indicating that leptin-mediated transcriptional activation of POMC promoter was dependent on SP1.

### Example 6

### Leptin-Mediated POMC Promoter Activity Requires Direct Interaction of STAT3 and SP1

The lack of STAT3 binding consensus sequence in the DNA element between −138 and −88 suggested that STAT3 regulation of POMC promoter activity was through a way other than direct STAT3-DNA interaction, i.e. STAT3 acted through an intermediate protein to mediate leptin action. As leptin-induced POMC promoter activation was dependent on SP1, we hypothesized that STAT3 regulated POMC promoter through its interaction with SP1. Co-IP using SP1 antibody resulted in abundant phospho-STAT3 signal in samples from leptin-treated, but not control 293-OBRb cells, whereas the control antibody did not pull down phospho-STAT3 from either leptin-treated or control cells (FIG. 6A). These data indicated that SP1 could bind to phospho-STAT3 specifically, and further suggested that STAT3 could act through SP1 to mediate leptin regulation of POMC promoter activity.

Discussion

Previous studies linked two putative STAT3 binding sites (−361 to −353, and −76 to −68) and one FoxO1 binding site (−375 to −370) to POMC expression (13, 22). In this study, however, deletion of these STAT3 binding sites (mutant 1, 4, and 9, FIG. 4), or FoxO1 binding site (mutant 4, FIG. 4) had little effect on leptin regulation of POMC promoter activity. Furthermore, SP1, but not STAT3 or FoxO1, was able to complex with the 51 bp DNA fragment essential for leptin regulation, suggesting that phosphorylated STAT3 enhance POMC promoter activity through a mechanism that requires SP1-POMC promoter complex, instead of a direct STAT3-POMC promoter interaction. SP1 is a constitutive transcription factor, and has been reported to serve as an intermediate in STAT3 regulation of gene expression (30-32), e.g. STAT3 mediates IL-6 induced VEGF promoter activity by interacting with SP1-DNA complex (30). Together with this study, these studies suggest an alternative mechanism to the established direct STAT3-DNA interaction in hormone/cytokine signaling, ie. STAT3 may regulate gene expression through its interaction with SP1-DNA complex (FIG. 7A).

### Example 7

### FoxO1 Inhibits STAT3-SP1 Interaction

Inhibition of STAT3-mediated leptin regulation of POMC promoter activity by FoxO1 occurred at a step downstream of STAT3 translocation into the nucleus (FIG. 3) and leptin action required a direct interaction of STAT3 and SP1. Whether FoxO1 could interfere with STAT3-SP1 complex formation, and thus prevent STAT3 from acting on the POMC promoter was tested.

To test whether FoxO1 could bind to STAT3, co-IP was performed on samples from 293-OBRb cells. FoxO1 was specifically coimmunoprecipitated in samples treated with antibody (anti-Flag) against Flag-tagged STAT3, but not the control antibody (FIG. 6B). Conversely, STAT3 was pulled down in samples treated with antibody (anti-Myc) against Myc tagged FoxO1, but not the control antibody (FIG. 6C). Thus, the two-way co-IP experiments confirmed FoxO1-STAT3 binding.

Whether increasing amount of FoxO1 could reduce, and even abolish SP1 binding to STAT3 was tested by co-IP of 293-OBRb cells that were transfected with increasing amount of FoxO1 cDNA. The ability of STAT3 antibody to pull down SP1 was inhibited by FoxO1, and STAT3-SP1 binding was undetectable at high FoxO1 expression levels (FIG. 6D). Together, these data demonstrated that FoxO1 could prevent STAT3-SP1 complex formation by binding to STAT3.

### Example 8

### Identification of Key FoxO1 Sequences Essential for STAT3 Interaction

FoxO1 is a 652 amino acid protein. To identify the FoxO1 sequences essential for STAT3 interaction, a series of C-terminal deletion constructs were made and tested their interaction with STAT3 by coimmunoprecipitation: FoxO1⁽¹⁻¹⁶⁷⁾ and other longer FoxO1 mutants were able to bind to STAT3, while FoxO1⁽¹⁻¹²³⁾ failed to bind to STAT3, suggesting that the region between amino acid residues 123-167 is important for STAT3 interaction.

A deletion construct, FoxO1⁽¹⁻¹²³⁾⁻⁽¹⁶⁸⁻⁶⁵²⁾ was made which does not contain the region identified in the previous C-terminal deletion constructs. As a control, a FoxO1 mutant was made that does not contain the region between 168-241, FoxO1⁽¹⁻¹⁶⁷⁾⁻⁽²⁴²⁻⁶⁵²⁾. Co-IP experiments using the above two deletion constructs confirmed the C-terminal deletion results that the region between 124-167 amino acids is necessary for STAT3 interaction because the FoxO1⁽¹⁻¹²³⁾⁻⁽¹⁶⁸⁻⁶⁵⁴⁾ peptide was not able to bind STAT3, but FoxO1⁽¹⁻¹⁶⁷⁾⁻⁽²⁴²⁻⁶⁵²⁾ did bind STAT3.

Discussion

In summary, these data demonstrate that 1) Phospho-STAT3 activates POMC promoter in response to leptin signaling through a mechanism that requires the SP1 binding site in the POMC promoter; 2) leptin action can be inhibited by FoxO1 at a step downstream of STAT3 phosphorylation and translocation into the nucleus; 3) FoxO1 binds to STAT3 and prevents STAT3 from interacting with the SP1-POMC promoter complex and consequently inhibits STAT3-mediated leptin action 4) FoxO1 binding to STAT3 requires residues within the 124-167 region. These data provide a potential mechanism for leptin resistance, in which an increased FoxO1 antagonizes STAT3-mediated leptin signaling by interfering with STAT3 SP1-target gene promoter complex formation.

In light of these data, the inventors have proposed a model of a potential mechanism of how FoxO1 inhibits leptin regulation of POMC promoter: With increasing amounts of FoxO1 expression, FoxO1 binds to phosphorylated STAT3 in the nucleus (via amino acid residues in the 124-167 region), and prevents STAT3 from interacting with the SP1-POMC promoter complex and consequently inhibits STAT3 mediated leptin activation of POMC promoter (FIG. 7B).

References

1. Zhang, Y., Proenca, R., Maffei, M., Barone, M., Leopold, L., and Friedman, J. M. (1994) Nature 372(6505), 425-432

2. Banks, W. A., Kastin, A. J., Huang, W., Jaspan, J. B., and Maness, L. M. (1996) Peptides 17(2),305-311

3. Cowley, M. A., Smart, J. L., Rubinstein, M., Cerdan, M. G., Diano, S., Horvath, T. L., Cone, R. D., and Low, M. J. (2001) Nature 411(6836), 480-484

4. Gong, L., Yao, F., Hockman, K., Heng, H. H., Morton, G. J., Takeda, K., Akira, S., Low, M. J., Rubinstein, M., and Mackenzie, R. G. (2008) Endocrinology

5. Vaisse, C., Halaas, J. L., Horvath, C. M., Darnell, J. E., Jr., Stoffel, M., and Friedman, J. M. (1996) Nat Genet 14(1), 95-97

6. Friedman, J. M., and Halaas, J. L. (1998) Nature 395(6704), 763-770

7. Bates, S. H., Steams, W. H., Dundon, T. A., Schubert, M., Tso, A. W., Wang, Y., Banks, A. S., Lavery, H. J., Haq, A. K., Maratos-Flier, E., Neel, B. G., Schwartz, M. W., and Myers, M. G., Jr. (2003) Nature 421(6925), 856-859

8. Schwartz, M. W., Peskind, E., Raskind, M., Boyko, E. J., and Porte, D., Jr. (1996) Nat Med 2(5),589-593

9. Myers, M. G., Cowley, M. A., and Munzberg, H. (2008) Annu Rev Physiol 70, 537-556

10. El-Haschimi, K., Pierroz, D. D., Hileman, S. M., Bjorbaek, C., and Flier, J. S. (2000) J Clin Invest 105(12), 1827-1832

11. Martin, T. L., Alquier, T., Asakura, K., Furukawa, N., Preitner, F., and Kahn, B. B. (2006) J Biol Chem 281(28), 18933-18941

12. Kim, M. S., Pak, Y. K., Jang, P. G., Namkoong, C., Choi, Y. S., Won, J. C., Kim, K. S., Kim, S. W., Kim, H. S., Park, J. Y., Kim, Y. B., and Lee, K. U. (2006) Nat Neurosci 9(7), 901-906

13. Kitamura, T., Feng, Y., Kitamura, Y. I., Chua, S. C., Jr., Xu, A. W., Barsh, G. S., Rossetti, L., and Accili, D. (2006) Nat Med 12(5), 534-540 14. Barthel, A., Schmoll, D., and Unterman, T. G. (2005) Trends Endocrinol Metab 16(4), 183-189

15. Hirota, K., Daitoku, H., Matsuzaki, H., Araya, N., Yamagata, K., Asada, S., Sugaya, T., and Fukamizu, A. (2003) J Biol Chem 278(15), 13056-13060

16. Cone, R. D. (2005) Nat Neurosci 8(5), 571-578

17. Mizuno, T. M., Kleopoulos, S. P., Bergen, H. T., Roberts, J. L., Priest, C. A., and Mobbs, C. V. (1998) Diabetes 47(2), 294-297

18. Huang, X. F., Xin, X., McLennan, P., and Storlien, L. (2004) Diabetes Obes Metab 6(1), 35-44

19. Munzberg, H., Huo, L., Nillni, E. A., Hollenberg, A. N., and Bjorbaek, C. (2003) Endocrinology 144(5), 2121-2131

20. Zhang, T., Kee, W. H., Seow, K. T., Fung, W., and Cao, X. (2000) Mol Cell Biol 20(19), 7132-7139

21. Ge, H., Huang, L., Pourbahrami, T., and Li, C. (2002) J Biol Chem 277(48), 45898-45903

22. Jeannotte, L., Trifiro, M. A., Plante, R. K., Chamberland, M., and Drouin, J. (1987) Mol Cell Biol 7(11), 4058-4064

23. Suske, G. (1999) Gene 238(2), 291-300

24. Campfield, L. A., Smith, F. J., Guisez, Y., Devos, R., and Burn, P. (1995) Science 269(5223),546-549

25. Pelleymounter, M. A., Cullen, M. J., Baker, M. B., Hecht, R., Winters, D., Boone, T., and Collins, F. (1995) Science 269(5223), 540-543

26. Enriori, P. J., Evans, A. E., Sinnayah, P., Jobst, E. E., Tonelli-Lemos, L., Billes, S. K., Glavas, M. M., Grayson, B. E., Perello, M., Nillni, E. A., Grove, K. L., and Cowley, M. A. (2007) Cell Metab 5(3), 181-194

27. Steppan, C. M., Bailey, S. T., Bhat, S., Brown, E. J., Banerjee, R. R., Wright, C. M., Patel, H. R., Ahima, R. S., and Lazar, M. A. (2001) Nature 409(6818), 307-312

28. Munzberg, H., Flier, J. S., and Bjorbaek, C. (2004) Endocrinology 145(11), 4880-4889

29. Bjorbaek, C., El-Haschimi, K., Frantz, J. D., and Flier, J. S. (1999) J Biol Chem 274(42), 30059-30065

30. Loeffler, S., Fayard, B., Weis, J., and Weissenberger, J. (2005) Int J Cancer 115(2), 202-213

31. Lin, S., Saxena, N. K., Ding, X., Stein, L. L., and Anania, F. A. (2006) Mol Endocrinol 20(12), 3376-3388

32. Li, H., Liang, J., Castrillon, D. H., DePinho, R. A., Olson, E. N., and Liu, Z. P. (2007) Mol Cell Biol 27(7), 2676-2686

33. Kortylewski, M., Feld, F., Kruger, K. D., Bahrenberg, G., Roth, R. A., Joost, H. G., Heinrich, P. C., Behrmann, I., and Barthel, A. (2003) J Biol Chem 278(7), 5242-5249

1. A method for identifying modulators of the interaction of STAT3 and SP1, the method comprising:
(a) providing a STAT3 polypeptide;
(b) providing an SP1 polypeptide;
(c) providing a FoxO1 polypeptide;
(d) contacting STAT3 and SP1 polypeptides in the presence of the FoxO1 polypeptide and a test compound; and
(d) detecting binding of STAT3 and SP1;
wherein the test compound is capable of binding a polypeptide comprising the peptide of SEQ ID NO: 1, or a peptide comprising at least 60% sequence identity to SEQ ID NO: 1.

2. A method for identifying modulators of the interaction of STAT3 and SP1, the method comprising:
(a) providing a STAT3 polypeptide;
(b) providing an SP1 polypeptide;
(c) contacting STAT3 and SP1 polypeptides in the presence of a test compound; and
(d) detecting binding of STAT3 and SP1;
wherein the test compound comprises a peptide comprising at least 60% sequence identity to the peptide of SEQ ID NO: 1, or a mimetic thereof.

3. A method of identifying compounds capable of suppressing appetite, the method comprising screening a test compound for the ability to bind to a peptide comprising SEQ ID NO: 1, or to a peptide having at least 60% sequence identity to SEQ ID NO: 1.

4. The method of any of claims 1 to 3 wherein binding of STAT3 and SP1 is complex formation.

5. The method of any preceding claim further comprising:
(e) testing whether the test compound mediates STAT3 SP1 mediated gene expression.

6. An appetite repressor or enhancer identified by the methods of any one of claims 1 to 3.

7. A medicament comprising an appetite repressor or enhancer of claim 6.

8. A method of identifying modulators of the interaction between STAT3 and SP1 comprising
(a) providing a cell comprising a STAT3 polypeptide, an SP1 polypeptide, a FoxO1 polypeptide and a STAT3 responsive promoter which is operably linked to a reporter gene;
(b) providing a test compound which is capable of binding to the peptide of SEQ ID NO: 1; and
(c) detecting expression of the reporter gene.

9. The method of claim 8 further comprising the step of:
(d) comparing expression of the reporter gene in step (c) to expression in the absence of the test compound

10. The method of claim 8 or 9 wherein the reporter gene is luciferase.

11. The method of any one of claims 8 to 10 further comprising the step of adding leptin.

12. The method of any one of claims 8 to 11 wherein the cell over expresses a leptin receptor.

13. The method of any one of claims 8 to 12 wherein the cell is a HEK293 cell.

14. A polypeptide comprising at least 60% sequence identity to SEQ ID NO: 1.

15. The polypeptide of claim 14, comprising at least 75% sequence identity to SEQ ID NO: 1.

16. The polypeptide of claim 15 comprising at least 90% sequence identity to SEQ ID NO: 1.

17. A polypeptide according to any one of claims 14 to 16 which comprises between 3 and 100 amino acids.

18. A polypeptide according to any one of claims. 14 to 16 which comprises between 3 and 44 amino acids.

19. A mimetic of the polypeptide according to SEQ ID NO: 1 which is capable of disrupting the interaction between STATS and SP1.

20. The polypeptide of any of claims 14 to 18, or the mimetic of claim 19, for use in the manufacture of a medicament for the repression or stimulation of appetite.
